(19) United States
(12) Patent Application Publication
(10) Pub. No.: US 2011/0217396 A1
(43) Pub. Date: Sep. 8, 2011

(54) Herbal Formulations for Controlling Blood Glucose Levels in Patients with Diabetes

(75) Inventor: Mario Dominik Oldani, Ameide (NL)

(73) Assignee: ATP MARKETING & PROMOTION AG, Niederurnen (CH)

(21) Appl. No.: 12/674,255

(22) PCT Filed: Aug. 21, 2007
(86) PCT No.: PCT/EP2007/007426

§ 371 Date: Feb. 19, 2010

(51) Int. Cl. A61K 36/54; A61P 3/10

(52) U.S. Cl. 424/739

(57) ABSTRACT

This invention relates to a composition and method for controlling blood glucose levels in patients with diabetes. An object of the present invention is to provide new herbal formulations for the treatment of diabetes, especially type 2 diabetes. In particular, the preparation and administration of herbal formulations comprising cinnamon and Gymnema sylvestre extract are described herein. An herbal formulation comprising Gymnema sylvestre extract, cinnamon extract, hydroxypropylcellulose, magnesium stearate, calcium phosphate, gelatine, and iron and titanium oxide exhibited improved control of type 2 diabetes as compared to the placebo group in clinical trials.

The present invention relates to herbal formulations for controlling blood glucose levels in a patient with diabetes and the use thereof for treating diabetes. The formulations according to the present invention are particularly useful for treating patients with diabetes type 2.

Diabetes mellitus is becoming an increasing public health concern world wide and especially in Europe and the United States. The disease is causing substantial morbidity, mortality, and long-term complications and is a major risk factor for the development of cardiovascular diseases.

Diabetes mellitus is the common denominator used to indicate a group of metabolic diseases characterized by high blood sugar (glucose) levels, usually resulting from defects in insulin secretion, or action, or both.

In diabetes mellitus, also commonly referred to as diabetes, elevated levels of blood glucose, also designated as hyperglycemia, lead to secretion of glucose into the urine by the kidneys.

Normally, blood glucose levels are tightly controlled by insulin, a hormone produced by the pancreas. Insulin lowers the blood glucose level. When the blood glucose elevates (for example after eating food), insulin is released from the pancreas to normalize the glucose level. In patients with diabetes, the absence or insufficient production of insulin causes hyperglycemia. Diabetes is a chronic medical condition, meaning, although it can be controlled, it lasts a lifetime.

There are two major types of diabetes, designated type 1 and type 2. Type 1 diabetes is also designated insulin dependent diabetes mellitus (IDDM), or juvenile onset diabetes mellitus. In type 1 diabetes, the pancreas undergoes an autoimmune attack by the body itself, and, as a consequence, the body is rendered incapable of making insulin.

Type 2 diabetes is also referred to as non-insulin dependent diabetes mellitus (NIDDM), or adult onset diabetes mellitus (AODM). In type 2 diabetes, patients can still produce insulin, but do so relatively inadequately for their body's needs.

The major goal in treating diabetes is controlling elevated blood sugar (glucose) without causing abnormally low levels of the blood sugar. Type 1 diabetes is treated with insulin, exercise, and a diabetic diet. Type 2 diabetes is first treated with weight reduction, a diabetic diet, and exercise. When these measures fail to control the elevated blood sugar, oral medications are used. If oral medications are still insufficient, insulin medications are considered.

Besides the treatment with insulin medications, it has been found that in especially diabetes type 2 patients, botanically based products can improve glucose metabolism and the overall condition of individuals not only by hypoglycemic (blood sugar reducing) effects, but also by improving lipid metabolism, antioxidant status and capillary function.

For example, cinnamon has been shown to reduce serum glucose, triglyceride, total cholesterol and LDL cholesterol levels especially in patients with type 2 diabetes. It is believed that, amongst others, the beneficial effects of cinnamon are obtained by decreasing the insulin resistance while increasing the insulin sensitivity providing an improved glucose uptake in the body cells.

Another example of a diabetes influencing herb is Gymnema sylvestre. The plant is a woody climber that grows in the tropical forests of central and southern India. Studies of an ethanol leaf extract, GS4, in diabetic rat and rabbit models have reported regeneration of islets of Langerhans, decreases in blood glucose, and increases of serum insulin.

The mechanism of action of this herb is unknown. However, postulated theories include an increase in insulin release through cell permeability, increase in beta-cell number and stimulation of beta-cell function.

Further, beneficial effects on especially diabetes type 2 have been reported for the herbs Coccinia indica, Ginseng species, Allium species, Ocimum sanctum, Trigonella foenum graecum, Bauhinia forficata and Mycria uniflora, Ficus carica, Opuntia streptacantha, Silibum marianum, Momordica charantia and Aloe vera.

Although at least some formulations of the above herbs have been clinically shown to be effective with respect to glycemic control in diabetic patients, and especially in diabetes type 2 patients, there still remains a need for other herb based formulations for the treatment of diabetes and especially diabetes type 2.

Therefore, an object of the present invention is to provide new herbal formulations for the treatment of diabetes, and especially diabetes type 2.

According to the present invention, this object is met by providing herbal formulations comprising cinnamon and Gymnema sylvestre extract.

Surprisingly, it was found by the present inventors that formulations comprising cinnamon and Gymnema sylvestre extract provide a synergistic effect in diabetic patients, and especially in type 2 diabetic patients, with respect to, amongst others, glycemic (blood sugar) control.

In a preferred embodiment of the present invention, the formulation comprises a cinnamon inner bark dry extract.

Preferably, the cinnamon inner bark dry extract is equivalent to 100 to 1000 mg, such as 100, 200, 300, 400, 500, 600, 700, 800, 900, 1000 mg daily dose, although doses higher than 1 gram cinnamon daily are contemplated within the scope of the present invention such as doses of 1, 2, 3, 4, 5, 6, 7, 8, 9, or 10 grams.

More preferably, the cinnamon inner bark dry extract is equivalent to 100 to 600, such as 100, 200, 300, 400, 500, 600 mg daily dose. Most preferably, the cinnamon inner bark dry extract is equivalent to at least 200 mg, such as 200, 300, 400, 500, 600, 700, 800, 900, or 1000 mg daily dose.

In a preferred form, the cinnamon is provided as a nebulisate 10 to 12:1, such as for example is sold under the trade name Diebacinn (Cinnamomum Zeylanicum Ness, water soluble extract).

The above referred daily dose of cinnamon in the formulation according to the present invention can be a single dose or a multiple dose such as for example a 3 times daily dose.

The therapeutically effective dose of cinnamon is dependent on the age, sex, weight, general health condition, the severity of the diabetes and other conditions such as cardiovascular disease, metabolism, blood pressure, etc. Provided with the above information, the skilled person will be able, without undue experimentation, to determine the therapeutically effective daily dose of cinnamon and its doses regime.

The formulation according to the present invention comprises a Gymnema sylvestre extract such as for example an extract comprising gymnemic acids such as, although not limiting, gymnemic acid I, gymnemic acid II, gymnemic acid III, etc. A particularly preferred Gymnema sylvestre extract is GS4, a standardized Gymnema sylvestre extract comprising 25% gymnemic acids.

Preferably, the Gymnema sylvestre extract is equivalent to 100 to 800 mg daily dose of a 25% Gymnema sylvestre extract, such as 100, 200, 300, 400, 500, 600, 700, or 800 mg, although doses lower or higher than the above range are contemplated within the scope of the present invention.

More preferably, the Gymnema sylvestre extract is equivalent to 100 to 600 mg daily dose of a 25% Gymnema sylvestre extract, such as 100, 200, 300, 400, 500, or 600 mg daily dose.

The above referred daily dose of Gymnema sylvestre extract in the formulation according to the present invention can be a single dose or a multiple dose such as for example a 3 times daily dose.

The therapeutically effective dose of Gymnema sylvestre extract is dependent on the age, sex, weight, general health condition, the severity of the diabetes and other conditions such as cardiovascular disease, metabolism, blood pressure, etc. Provided with the above information, the skilled person will be able, without undue experimentation, to determine the therapeutically effective daily dose of Gymnema sylvestre extract and its dose regime.

In a particularly preferred embodiment, the formulation according to the present invention also comprises micronutrients and/or trace elements. Suitable examples of such compounds are chromium species, magnesium, vitamin E, L-Carnitine, vanadium, and alpha-lipoic acid also known as thioctic acid. These compounds are known to have an effect in diabetic patients and can therefore be suitably combined with the herbal formulations according to the present invention.

The formulations according to the present invention may be administered in standard manner for the disease condition that it is desired to treat, for example by oral, topical, parenteral, buccal, nasal, vaginal or rectal administration or by inhalation or insufflation. Preferred however is oral administration.

For these purposes, the compounds according to the present invention may be formulated by means known in the art into the form of, for example, tablets, pellets, capsules, aqueous or oily solutions, suspensions, emulsions, creams, ointments, gels, nasal sprays, suppositories, finely divided powders or aerosols or nebulizers for inhalation, and for parenteral use (including intravenous, intramuscular or infusion) sterile aqueous or oily solutions or suspensions or sterile emulsions.

However, preferred are capsules such as soft or hard gelatine capsules. In this preferred embodiment, the capsules are preferably filled with a particulate or microparticulate of the cinnamon and Gymnema sylvestre extracts according to the present invention.

The formulations according to the present invention can also comprise carriers and additives which are commonly used in the pharmaceutical field. Such carriers and additives can for example be:

Solvents such as purified water, water for injection, physiological saline, peanut oil, ethanol, and glycerin;

Carriers such as starch, lactose, glucose, sucrose, microcrystalline cellulose, methyl cellulose, calcium sulfate, calcium carbonate, talc, titanium oxide, trehalose, and xylitol;

Coating agents such as sucrose, gelatin, and cellulose acetate phthalate; basis: vaseline, vegetable oil, macrogol, oil in water type emulsion base, water in oil type emulsion base;

Binders such as starch and derivatives thereof, cellulose and derivatives thereof, naturally-occurring high molecular compounds such as gelatin, sodium alginate, tragacanth, acacia and the like, synthetic high molecular compounds such as polyvinyl pyrrolidone, dextrin, and hydroxypropyl starch;

Lubricants such as stearic acid and salts thereof, talc, wax, wheat starch, macrogol, hydrogenated vegetable oil, sucrose fatty acid ester, and polyethylene glycol;

Disintegrants such as starch and derivatives thereof, gelatin, gelatin powder, sodium bicarbonate, cellulose and derivatives thereof, calcium carmellose, hydroxypropyl starch, carboxymethyl cellulose and salts and cross-linked materials thereof, and low-substituted types of hydroxypropyl cellulose;

Solution adjuvants such as cyclodextrin, ethanol, propylene glycol, and polyethylene glycol; suspending agents such as acacia, tragacanth, sodium alginate, aluminum monostearate, citric acid, and various surfactants;

Viscosity-increasing agents such as sodium carmellose, polyvinyl pyrrolidone, methyl cellulose, hydroxypropylmethyl cellulose, polyvinyl alcohol, tragacanth, acacia, and sodium alginate;

Emulsifying agents such as acacia, cholesterol, tragacanth, methyl cellulose, various surfactants, lecithin;

Stabilizers such as sodium hydrogensulfite, ascorbic acid, tocopherol, chelating agent, inert gas, and reducing substance;

Buffers such as sodium hydrogenphosphate, sodium acetate, and boric acid;

Tonicity agents such as sodium chloride and glucose;

Soothing agents such as procaine hydrochloride, lidocaine, benzyl alcohol;

Preservatives such as benzoic acid and salts thereof, para-oxybenzoic acid esters, chlorobutanol, invert soap, benzyl alcohol, phenol, and thimerosal; flavoring agents such as sucrose, saccharin, glycyrrhiza extract, sorbitol, xylitol, and glycerin;

Aromas such as orange peel tincture and rose oil; and

Colorants: water-soluble food pigment, and lake pigment.

In a particularly preferred embodiment of the present invention, the present formulation comprises:
Gymnema sylvestre extract
Cinnamon (ZN112)
hydroxypropylcellulose
Magnesium stearate; and
dicalcium phosphate

Preferably, the above formulation is incorporated in a gelatine capsule for oral administration providing a single dose of the active ingredients of:
Gymnema sylvestre extract 134 mg; and
Cinnamon (ZN112) 112 mg

Although the invention has been described in some detail above by referring to specific preferred embodiments, it should be understood that the scope of the present invention, which solely defined by the appended claims, is not limited to these embodiments. The skilled person will appreciate that modifications and adaptations can be made to the present invention without deviating from the inventive concept of the invention.

### EXAMPLE

Metabolic effects of a combination preparation of Diabecinn (oral cinnamon extract) and Gymnema Sylvestre in diabetes type 2, a placebo-controlled randomized clinical trial (DiabGym trial).

### Objective

An objective of the randomized, placebo-controlled trial was to determine the effects of the combination of cinnamon and Gymnema Sylvestre on the production of insulin and subsequently the level of the fasting glucose in type 2 diabetic patients.

### Study Design

The study was a double blind randomized parallel clinical trial with a trial duration of 3 weeks.

### Trial Population

Twenty type 2 diabetic patients participated, age 35-76 years. Exclusion criteria were pregnancy or breast-feeding at the time of trial and use of oral anti-coagulants.

### Intervention

Patients were randomized to DiabGym or matching placebo. Oral antidiabetic medication and insulin use was left unchanged during the trial, unless hypoglycemia necessitated down titration. Antilipemic medication was left unchanged during the trial.

### Endpoints

Primary outcome measure were differences between the treatment groups in baseline corrected insulin and C-peptide at the end of the treatment period. Main secondary outcome measure was fasting glucose.

### Burden for the Participants

There were eight study visits, two scheduled telephonic contacts and blood was drawn eight times. There are no known side effects of cinnamon and Gymnema Sylvestre. Improvement of glycemic abnormalities were observed for those randomized to active substances.

### Study Design

The study was a double blind randomized parallel clinical trial. Trial duration was 3 weeks. Twenty type 2 diabetic patients on either diet, oral medication, insulin or a combination thereof, with or without antilipaemic treatment, were selected and screened.

Hereafter, patients were treated with a combination of Cinnamon and Gymnema Sylvestre or matched placebo for a 3 week period, on top of their current therapeutic regimen, which will remain unchanged for the duration of the trial period, unless hypoglycemia occurs.

The group of twenty volunteers were divided into four subgroups:
Group A received once daily a dose of DiabGym.
Group B received three times daily a dose of DiabGym.
Group C received five times daily a dose of DiabGym.
Group D received once daily a dose of a placebo.

### Trial Population

### Inclusion Criteria

Type 2 diabetes patients on any type of treatment

Age 35-76 years

### Exclusion Criteria

Pregnancy or breast-feeding at the time of trial

Use of oral anti-coagulants

### Outcome Measures

Primary outcome measure at the end of the treatment period was differences between the treatment groups in baseline corrected insulin production and fasting glucose in relation to the dose given. Main secondary outcome measure was measurement of the possible development of insulin antibodies. Other secondary outcome measures included:

Body weight

CRP

### Safety Measures Included:

ASAT, ALAT, bilirubin

Creatinin and other safety measures

### Treatment

Patients were randomized to DiabGym (1, 3 or 5 times daily a dose) or matching placebo. Oral antidiabetic medication and insulin use was left unchanged during the trial, unless hypoglycemia necessitates down titration. Antilipaemic medication was left unchanged during the trial.

### Adverse Events

Adverse events were recorded according to GCP practice. Severe Adverse effects definition follows the standard GCP definition: any adverse event resulting in:
Death
A life-threatening experience
Subject hospitalization or prolongation of existing hospitalization
A persistent or significant disability/incapacity
A congenital anomaly/birth defect.

Important medical events that may not result in death, be life-threatening, or require hospitalization were considered an SAE when, based upon appropriate medical judgement, they jeopardized the subject and required medical or surgical intervention to prevent one of the outcomes listed in this definition.

### Product Information

Capsules filled with a combination of cinnamon extract ZN 112 and Gymnema Sylvestre, or matching placebo were provided by OTC Pharma International B.V., Gorinchem, the Netherlands. The composition of each single dose was:
Cinnamon: Cinnamomum Zeylanicum Ness, water soluble extract of cinnamon, 10-12:1 (ZN112)
Gymnema Sylvestre: Gymnema Sylvestre extract, 25%

### Resulting in:

Gymnema sylvestre extract 134 mg
Water soluble extract of cinnamon (ZN112) 112 mg
Hydroxypropylcellulose 30.4 mg
Magnesium stearate 2 mg
Calcium phosphate 25.6 mg
Gelatine 90.8 mg
Iron and titanium oxide

### Clinical Assessments

The patient were be seen at U-Diagnostics BV in Utrecht for study visits.

Visit 0 included: obtaining informed consent, before any other trial related activity is performed, establishment of in- and exclusion criteria.

At Visit 1 (within three weeks of Visit 0) a fasting blood sample, blood pressure and body weight were taken, the patient were randomized and medication was issued.

In the following three weeks, an additional 5 visits were scheduled. Each time a fasting blood sample was taken and a questionnaire was filled in.

The closing visit was three weeks later than visit 1. Also a fasting blood sample were taken.

### Results

It was shown that the treatment groups receiving the capsules filled with a combination of cinnamon extract ZN 112 and Gymnema Sylvestre showed an improved control of type 2 diabetes as compared to the placebo group. This result is surprising and cannot be attributed to the combined known separate activities of the two active ingredients.

1-11. (canceled)

12. An herbal formulation comprising cinnamon and Gymnema sylvestre extract.

13. The herbal formulation according to claim 12 comprising, as daily dose, 100 to 1000 mg equivalent cinnamon and 100 to 800 mg equivalent 25% Gymnema sylvestre extract.

14. The herbal formulation according to claim 12, further comprising micronutrients and/or trace elements.

15. The herbal formulation according to claim 12, further comprising pharmaceutically acceptable carriers and excipients.

16. The herbal formulation according to claim 12 formulated for oral administration.

17. The herbal formulation according to claim 12 comprising:
Gymnema sylvestre extract;
cinnamon (ZN112);
hydroxypropylcellulose;
magnesium stearate; and
dicalcium phosphate.

18. The herbal formulation according to claim 12, formulated for use as a medicament.

19. A method of using the herbal formulation according to claim 12 for the preparation of a medicament for the treatment of diabetes.

20. The method of claim 19, wherein the diabetes is type 2 diabetes.

21. A method for treating diabetes comprising administering the herbal formulation according to claim 12.

22. The method of claim 21, wherein the diabetes is type 2 diabetes.
